(19) United States
(12) Patent Application Publication
(10) Pub. No.: US 2011/0060413 A1
(43) Pub. Date: Mar. 10, 2011

(54) GUIDED BONE REGENERATION MEMBRANE AND MANUFACTURING METHOD THEREOF

(75) Inventors: Toshihiro KASUGA, Kiyosu (JP); Akiko Obata, Nagoya (JP); Kie Fujikura, Kobe (JP)

(73) Assignee: NATIONAL UNIVERSITY CORPORATION NAGOYA INSTITUTE OF TECHNOLOGY, Nagoya-shi (JP)

(21) Appl. No.: 12/876,665

(22) Filed: Sep. 7, 2010

(30) Foreign Application Priority Data
Sep. 10, 2009 (JP) 2009-208922

(51) Int. Cl. A61F 2/28; B29C 47/04

(52) U.S. Cl. 623/16.11; 264/466

(57) ABSTRACT

A guided bone regeneration membrane has a bilayer structure including a first nonwoven fabric layer and a second nonwoven fabric layer. The first nonwoven fabric layer includes a fibrous substance containing a biodegradable resin as a principal component and further containing a siloxane, and the second nonwoven fabric layer includes a fibrous substance containing a biodegradable resin as a principal component. The first and second nonwoven fabric layers are electrospun nonwoven fabric layers, and the fibrous substance constituting the second nonwoven fabric layer has an average diameter smaller than that of the fibrous substance constituting the first nonwoven fabric layer. Specifically, the fibrous substance constituting the second nonwoven fabric layer preferably has an average diameter of more than 0 μm and equal to or less than 5 μm.

### CROSS REFERENCE TO RELATED APPLICATION

This application is based upon and claims the benefit of Japanese Patent Application No. 2009-208922 filed on Sep. 10, 2009, the content of which are incorporated herein by reference.

### FIELD OF THE INVENTION

The present invention relates to a guided bone regeneration membrane and a manufacturing method thereof. The guided bone regeneration membrane is used in a guided bone regeneration (GBR) technique which is one of techniques for repairing bone defects and which is widely used in the field of orthopedic surgery and oral or maxillofacial surgery.

### RELATED ART OF THE INVENTION

Guided bone regeneration membranes are masking membranes that cover bone defect areas so as to prevent invasion of non-osteogenesis-contributed cells and tissues into the bone defect areas and to allow the bone to reconstruct by taking full advantage of self-regenerative power thereof. Guided bone regeneration techniques using these membranes cure bone defects by using a healing potential which the living body inherently has. The techniques are not complicated in their operative procedures and have given many satisfactory outcomes in, orthopedic surgery and oral surgery.

As one of such guided bone regeneration membranes, Japanese Unexamined Patent Application Publication (JP-A) No. 2009-61109 discloses a guided bone regeneration membrane having a bilayer structure including a first nonwoven fabric layer and a second nonwoven fabric layer, in which the first nonwoven fabric layer contains siloxane-containing calcium carbonate fine particles and a biodegradable resin (e.g., a poly(lactic acid)) as principal components; and the second nonwoven fabric layer contains a biodegradable resin (e.g., a polylactic acid)) as a principal component. This guided bone regeneration membrane is intended so that the first nonwoven fabric layer accelerates the bone regeneration due to the function of the siloxane as a factor for promoting osteogenesis, and the second nonwoven fabric layer prevents the invasion of non-osteogenesis-contributed cells and soft tissues into the bone defect areas.

### SUMMARY OF THE INVENTION

Materials for guided bone regeneration membranes require such flexibility (plasticity) as to deform along the shape of the affected area and to maintain its dimensions. This is because, when a guided bone regeneration membrane having low flexibility covers a bone defect area, there occurs a gap between the guided bone regeneration membrane and an area around the bone defect area, and a soft tissue will invade via the gap into the bone defect area. The invasion of the soft tissue via the gap impedes the bone regeneration, because cells constituting the soft tissue grow faster than osteoblasts.

For this reason, it is important for a second nonwoven fabric layer in a guided bone regeneration membrane having such a bilayer structure to have high flexibility so as to reliably prevent the invasion of non-osteogenesis-contributed cells and soft tissues into the bone defect areas.

This is true not only for such a guided bone regeneration membrane having a first nonwoven fabric layer composed of a fibrous substance containing calcium carbonate fine particles bearing a siloxane dispersed therein and a biodegradable resin as principal components, but also for a guided bone regeneration membrane having a first nonwoven fabric layer composed of a fibrous substance containing, as a principal component, a siloxane-containing biodegradable resin and containing no calcium carbonate fine particles.

Under these circumstances, an object of the present invention is to improve the bone regeneration ability of a guided bone regeneration membrane having the above-mentioned bilayer structure by improving the flexibility of the second nonwoven fabric layer.

To achieve the object, the present invention provides, in an embodiment, a guided bone regeneration membrane having a bilayer structure including a first nonwoven fabric layer and a second nonwoven fabric layer, the first nonwoven fabric layer including a first fibrous substance containing a biodegradable resin as a principal component and further containing a siloxane, and the second nonwoven fabric layer including a second fibrous substance containing a biodegradable resin as a principal component, in which the first and second nonwoven fabric layers are both layers of electrospun nonwoven fabrics, and the second fibrous substance constituting the second nonwoven fabric layer has an average diameter smaller than that of the first fibrous substance constituting the first nonwoven fabric layer.

First of all, the guided bone regeneration membrane according to the embodiment of the present invention is intended so that the first nonwoven fabric layer containing the siloxane accelerates the bone regeneration, and the second nonwoven fabric layer prevents the invasion of non-osteogenesis-contributed cells and soft tissues into the bone defect areas, as with the technique disclosed in Patent Document 1.

In addition, according to the present invention, the second fibrous substance constituting the second nonwoven fabric layer has an average diameter controlled to be smaller than that of the first fibrous substance constituting the first nonwoven fabric layer, which improves the flexibility of the second nonwoven fabric layer as compared to the case where a fibrous substance constituting a second nonwoven fabric layer has an average diameter equal to that of a fibrous substance constituting a first nonwoven fabric layer. This is because such a fibrous substance containing a biodegradable resin as a principal component has increasing flexibility with a decreasing diameter thereof.

The guided bone regeneration membrane according to the present invention can therefore have improved flexibility of the second nonwoven fabric layer and, when used to cover the bone defect area, can reliably prevent a gap between itself and an area around the bone defect area, and can exhibit higher bone regeneration ability.

When the first and second nonwoven fabric layers are layers of electrospun nonwoven fabrics as in the present invention, space among fibers (fibrous substance) can be increased by increasing the diameter of the constitutive fibrous substance; and can be decreased by decreasing the diameter of the constitutive fibrous substance.

Accordingly, the first fibrous substance constituting the first nonwoven fabric layer is controlled to have a larger diameter so that the first nonwoven fabric layer can have larger space among the fibers (fibrous substance) to allow the invasion of osteogenesis-contributed cells into the first nonwoven fabric layer. This allows the growth of the osteogenesis-contributed cells in the first nonwoven fabric layer. On the other hand, the second fibrous substance constituting the second nonwoven fabric layer is controlled to have a smaller diameter so that the second nonwoven fabric layer can have smaller space among the fibers (fibrous substance). This prevents the invasion of non-osteogenesis-contributed cells and soft tissues thereinto.

More specifically, the second fibrous substance constituting the second nonwoven fabric layer preferably has an average diameter of more than 0 μm and equal to or less than 5 μm. This second nonwoven fabric layer can further satisfactorily prevent the invasion of non-osteogenesis-contributed cells and soft tissues into the bone defect area. The first fibrous substance constituting the first nonwoven fabric layer preferably has an average diameter of 10 μm or more and 20 μm or less. This first nonwoven fabric layer can further satisfactorily allow osteogenesis-contributed cells to invade thereinto.

The first fibrous substance constituting the first nonwoven fabric layer may further include calcium carbonate fine particles and integrally contain the siloxane as dispersed in the calcium carbonate fine particles. The biodegradable resin is preferably a polylactic acid) (hereinafter briefly referred to as PLA) or a copolymer thereof (copolymer of lactic acid with one or more other monomers).

When the first fibrous substance constituting the first nonwoven fabric layer further includes calcium carbonate fine particles and integrally contains the siloxane as dispersed in the calcium carbonate fine particles as mentioned above, the entire first nonwoven fabric layer has somewhat lower flexibility due to the presence of such stiff calcium carbonate fine particles, as compared to the case where the first nonwoven fabric layer contains no calcium carbonate fine particles. For this reason, the flexibility of the second nonwoven fabric layer is particularly important from the viewpoint of covering the affected area without a gap, and the present invention is particularly effectively adopted to the case where the first nonwoven fabric layer further includes calcium carbonate fine particles and integrally contains the siloxane as dispersed in the calcium carbonate fine particles.

The present invention further provides, in another embodiment, a method for manufacturing a guided bone regeneration membrane having a bilayer structure including a first nonwoven fabric layer and a second nonwoven fabric layer, the first nonwoven fabric layer including a first fibrous substance containing a biodegradable resin as a principal component and further containing a siloxane, and the second nonwoven fabric layer including a second fibrous substance containing a biodegradable resin as a principal component. The method includes the steps of forming the first nonwoven fabric layer through electrospinning; and forming the second nonwoven fabric layer through electrospinning, in which the step of forming the second nonwoven fabric layer through electrospinning is performed so that the second fibrous substance constituting the second nonwoven fabric layer has an average diameter smaller than that of the first fibrous substance constituting the first nonwoven fabric layer. The method according to the embodiment of the present invention shows the same advantageous effects as above.

Specifically, in the method according to the present invention, the second fibrous substance constituting the second nonwoven fabric layer is preferably formed so as to have an average diameter of more than 0 μm and equal to or less than 5 μm. The first fibrous substance constituting the first nonwoven fabric layer is preferably formed so as to have an average diameter of 10 μm or more and 20 μm or less. A fibrous substance further containing calcium carbonate fine particles and integrally containing the siloxane as dispersed in the calcium carbonate fine particles may be used as the first fibrous substance to constitute the first nonwoven fabric layer. A poly(lactic acid) or a copolymer thereof may be used as the biodegradable resin.

### BRIEF DESCRIPTION OF THE DRAWINGS

Other objects, features and advantages of the present invention will be understood more fully from the following detailed description made with reference to the accompanying drawings. In the drawings:

FIG. 1 is a scanning electron micrograph (SEM photograph) of fibers constituting a siloxane-containing polylactic acid) (Si-PLA) layer prepared as a first nonwoven fabric layer in Example 1;

FIG. 2 is a scanning electron micrograph of fibers constituting a PLA layer (5 μm in diameter) prepared as a second nonwoven fabric layer in Example 1;

FIG. 3 is a scanning electron micrograph of fibers constituting a PLA layer (1 to 2 μm in diameter) prepared as a second nonwoven fabric layer prepared in Example 1;

FIG. 4 is a graph showing how the pore sizes distribute in three nonwoven fabric layers composed of fibrous substances having different diameters;

FIG. 5 is a graph showing how deep cells invade into three nonwoven fabric layers composed of fibrous substances having different diameters;

FIG. 6 is a scanning electron micrograph of fibers constituting a siloxane-containing calcium carbonate (Si—CaCO₃)/PLA layer prepared as a first nonwoven fabric layer in Example 2; and

FIG. 7 is a scanning electron micrograph of fibers constituting a PLA layer prepared as a second nonwoven fabric layer in Example 2.

### DETAILED DESCRIPTION OF THE PREFERRED EMBODIMENTS

A guided bone regeneration membrane according to an embodiment of the present invention has a bilayer structure including a first nonwoven fabric layer and a second nonwoven fabric layer, in which the first nonwoven fabric layer is composed of a first fibrous substance containing a biodegradable resin as a principal component and further containing a siloxane; and the second nonwoven fabric layer is composed of a second fibrous substance containing a biodegradable resin as a principal component. This guided bone regeneration membrane is intended so that the first nonwoven fabric layer accelerates the bone regeneration due to the function of the siloxane as a factor for promoting osteogenesis, and the second nonwoven fabric layer prevents the invasion of non-osteogenesis-contributed cells and soft tissues into the bone defect areas.

The first and second nonwoven fabric layers are respectively layers of electrospun nonwoven fabrics formed through electrospinning. Typically in the electrospinning, while a high positive voltage is applied thereto, a spinning dope is sprayed to a negatively charged collector, during which a substance in the spinning dope forms fibers and is deposited.

A spinning dope for the formation of the first nonwoven fabric layer (hereinafter also referred to as “first spinning dope”) is a solution, in a solvent, of a substance containing a biodegradable resin as a principal component and further containing a siloxane; and a spinning dope for the formation of the second nonwoven fabric layer (hereinafter also referred to as “second spinning dope”) is a solution, in a solvent, of a substance containing a biodegradable resin as a principal component.

The biodegradable resin is preferably a poly(lactic acid) (PLA) or a copolymer between lactic acid and glycolic acid (copoly(lactic acid/glycolic acid); PLA/PGA). Exemplary other biodegradable resins usable herein include synthetic polymers such as polyethylene glycols (PEGs), polycaprolactones (PCLs), as well as copolymers among lactic acid, glycolic acid, ethylene glycol, and/or caprolactone; and natural polymers such as fibrin, collagens, alginic acids, hyaluronic acids, chitins, and chitosans.

A representative example of the first spinning dope for the formation of the first nonwoven fabric layer is a solution prepared by dissolving a poly(lactic acid) (PLA) in chloroform (CHCl₃) or dichloromethane and further mixing with an aqueous aminopropyltriethoxysilane (APTES) solution. The weight ratio of PLA to APTES in the solution may be from 1:0.01 to 1:0.5 and is preferably from 1:0.01 to 1:0.05. This is because, if APTES is added in an excessively large amount, most of APTES contained in the resulting fibers is dissolved out in early stages when the fibers are immersed in an aqueous solution, thus APTES does not act so effectively. The concentration of the poly(lactic acid) (molecular weight: about 20×10⁴ to about 30×10⁴ daltons (Da)) is preferably from 4 to 12 percent by weight for easy spinning. The first spinning dope may further contain dimethylformamide or methanol in an amount of up to about 50 percent by weight with respect to the weight of chloroform or dichloromethane, to carry out satisfactory spinning. The use of such spinning dope gives a first nonwoven fabric layer composed of a siloxane-containing biodegradable resin.

Another preferred example of the first spinning dope for the formation of the first nonwoven fabric layer is a solution, in a solvent, of a substance containing a biodegradable resin as a principal component and further containing a siloxane, in which the substance is prepared by preparing calcium carbonate fine particles containing a siloxane dispersed therein (Si—CaCO₃) typically by the method described in Japanese Unexamined Patent Application Publication (JP-A) No. 2008-100878 and mixing the Si—CaCO₃ in an amount up to 60 percent by mass with a poly(lactic acid). Yet another preferred example of the first spinning dope is a solution prepared by kneading a poly(lactic acid) and Si—CaCO₃ fine particles in a predetermined ratio using a heating kneader to give a composite; and dissolving the composite in a solvent. This technique is advantageous for uniform dispersion of the fine particles. The use of such spinning dope gives a first nonwoven fabric layer composed of a composite of a biodegradable resin and calcium carbonate fine particles containing a siloxane dispersed therein.

A representative example of the second spinning dope for the formation of the second nonwoven fabric layer is a solution of a poly(lactic acid) in chloroform (CHCl₃) or dichloromethane. The use of such spinning dope gives a second nonwoven fabric layer composed of a biodegradable resin.

Using an electrospinning apparatus, the first spinning dope is sprayed to form a first nonwoven fabric layer, and the second spinning dope is subsequently sprayed to form a second nonwoven fabric layer on the first nonwoven fabric layer to thereby give a guided bone regeneration membrane having a bilayer structure. Such a guided bone regeneration membrane having a bilayer structure can also be manufactured, for example, by initially forming a second nonwoven fabric layer and then forming a first nonwoven fabric layer, or by forming first and second nonwoven fabric layers separately and bonding the two layers.

A desired guided bone regeneration membrane can be prepared by appropriately setting spinning conditions such as the concentration, solvent type, and supply speed (feed rate) of the spinning dopes; spinning time; applied voltage; and distance between the nozzle and the collector. According to the embodiment of the present invention, the spinning conditions for the first and second nonwoven fabric layers are set so that the second fibrous substance constituting the second nonwoven fabric layer has an average diameter smaller than that of the first fibrous substance constituting the first nonwoven fabric layer. Specifically, in a preferred embodiment, the spinning conditions are set so that the first fibrous substance constituting the first nonwoven fabric layer has an average diameter of 10 μm or more and 20 μm or less, and the second fibrous substance constituting the second nonwoven fabric layer has an average diameter of more than 0 μm and equal to or less than 5 μm. The diameter of such a fibrous substance can be substantially controlled by the viscosity of a spinning dope, and the viscosity of the spinning dope in turn depends typically on the concentration of the spinning dope and the type and proportion of the solvent. Thus, a fibrous substance having a desired diameter is given typically by setting the concentration of the spinning dope, and the type and proportion of the solvent therein. In this connection, the average diameter of a fibrous substance can be determined typically through an electron microscopic observation.

As is described above, the second fibrous substance constituting the second nonwoven fabric layer has an average diameter controlled to be smaller than that of the first fibrous substance constituting the first nonwoven fabric layer, which improves the flexibility of the second nonwoven fabric layer as compared to the case where a fibrous substance constituting a second nonwoven fabric layer has an average diameter equal to that of a fibrous substance constituting a first nonwoven fabric layer. This is because such a fibrous substance containing a biodegradable resin as a principal component has increasing flexibility with a decreasing diameter thereof.

Accordingly, the guided bone regeneration membrane according to the present invention can have improved flexibility of the second nonwoven fabric layer and, when used to cover the bone defect area, can reliably prevent a gap between itself and an area around the bone defect area, and can exhibit higher bone regeneration ability.

When the first fibrous substance constituting the first nonwoven fabric layer is a composite of Si—CaCO₃ fine particles and a polylactic acid) as in the embodiment above, the first nonwoven fabric layer has lower flexibility and lower strength as compared to the case where the fibrous substance contains a siloxane-containing poly(lactic acid) alone and contains no CaCO₃ fine particles. The first nonwoven fabric layer with less flexibility and less strength may be likely to be broken to cause a gap when the guided bone regeneration membrane covers the affected area. The second nonwoven fabric layer in the guided bone regeneration membrane according to this embodiment, however, ensures covering of the affected area without a gap, because the second nonwoven fabric layer has higher flexibility.

When the first and second nonwoven fabric layers are formed using one electrospinning apparatus, space among fibers (i.e., pore size) can be increased by increasing the diameter of the fibrous substance; and it can be decreased by decreasing the diameter of the fibrous substance.

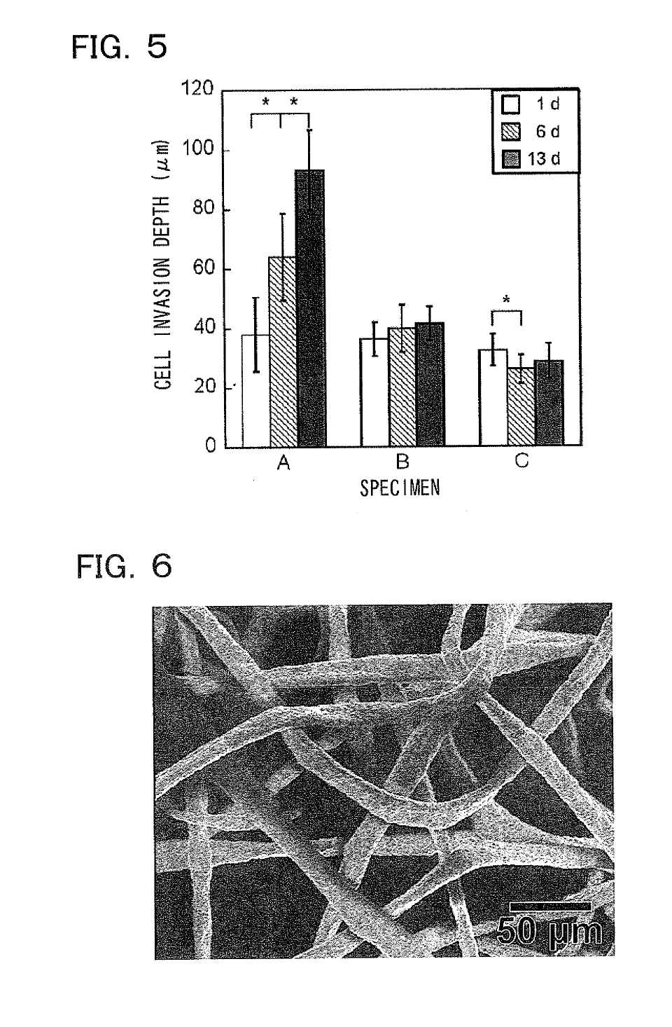

Thus, the space among fibers (fibrous substance) constituting the first nonwoven fabric layer is allowed to be larger than the size of cells per se to thereby allow cells (osteogenesis-contributed cells) to invade into the first nonwoven fabric layer, by allowing the first fibrous substance constituting the first nonwoven fabric layer to have an average diameter of 10 μm or more, as described in working examples mentioned below (see FIG. 5). The first fibrous substance has an average diameter of preferably 20 μm or less, because a fibrous substance formed with a regular electrospinning apparatus has an average maximum diameter of up to about 20 μm.

Independently, the space among fibers (fibrous substance) constituting the second nonwoven fabric layer is allowed to be smaller than the sizes of cells per se to thereby prevent the invasion of non-osteogenesis-contributed cells and soft tissues into the second nonwoven fabric layer, by allowing the second fibrous substance constituting the second nonwoven fabric layer to have an average diameter of 5 μm or less, as described in working examples mentioned below (see FIG. 5). In this connection, it is difficult for a currently-employed electrospinning apparatus to manufacture a fibrous substance having a diameter of less than 0.05 μm. However, the second fibrous substance has only to have an average diameter of more than 0 from the viewpoint of exhibiting the function thereof, and the manufacture of such a fibrous substance having a diameter of less than 0.05 μm will become possible by improvements in electrospinning apparatus in future.

Unlike the method according to the present invention, a second nonwoven fabric layer having a small pore size can also be formed by forming a second nonwoven fabric layer composed of a fibrous substance having an average diameter equal to that of a fibrous substance constituting a first nonwoven fabric layer, and pressing the formed second nonwoven fabric layer. This technique, however, requires the pressing step to allow the second nonwoven fabric layer to have a desired pore size. In addition, the second nonwoven, fabric layer, if formed under the same conditions as with the first nonwoven fabric layer, has a relatively inferior flexibility due to large average diameter of its constitutive fibrous substance, as compared to the case where the second fibrous substance has a smaller average diameter. Such flexibility of the second nonwoven fabric layer may often be lowered as a result of pressing.

In contrast, the manufacturing method according to the embodiment of the present invention can give a second nonwoven fabric layer having a desired pore size by setting spinning conditions in the step of forming the second nonwoven fabric layer through electrospinning so as to have a desired fiber diameter. In addition, the method according to this embodiment does not include a pressing step and thereby allows the second nonwoven fabric layer to have higher flexibility than the case where the second nonwoven fabric layer is subjected to pressing.

### EXAMPLES

The present invention will be illustrated in further detail with reference to several working examples below relating to the guided bone regeneration membrane and the method for manufacturing the same. It should be noted, however, that these examples are included merely to aid in the understanding of the present invention and are not to be construed to limit the scope of the present invention.

Raw materials used in the examples are as follows.

Poly(lactic acid) (PLA): One having a molecular weight of 20×10⁴ to 30×10⁴ Da, PURAC Biochem BV, Netherlands; or one having a molecular weight of 15×10⁴ to 17×10⁴ Da, Shimadzu Corporation, Japan

Chloroform (CHCl₃): Analytical grade reagent, purity 99.0% or more, Chemical Co., Ltd., Japan

γ-Aminopropyltriethoxysilane (APTES): TSL 8331, purity of 98% or more, GE Toshiba Silicones Co., Ltd., Japan

Siloxane-containing calcium carbonate (Si—CaCO₃): Vaterite containing a siloxane (2.9 percent by weight in terms of silicon ion) and prepared from slaked lime (Microstar T; purity 96% or more; Yabashi Industries Co., Ltd., Japan), methanol (analytical grade reagent; purity 99.8% or more; Kishida Chemical Co., Ltd., Japan), APTES, and carbon dioxide gas (high-purity liquefied carbon dioxide gas; purity 99.9%; Taiyo Kagaku Kogyo K. K., Japan)

### Example 1

A Si-PLA layer was prepared as a first nonwoven fabric layer. Independently, two PLA layers having different average diameters of constitutive fibrous substances (5 μm in diameter and 1 to 2 μm in diameter) were prepared each as a second nonwoven fabric layer.

Specifically, a spinning dope for the Si-PLA layer was prepared by dissolving 1.0 g of a poly(lactic acid) having a molecular weight of 15×10⁴ to 17×10⁴ Da in chloroform to give a 8% PLA solution, and blending the PLA solution with 0.075 g of a 67% APTES aqueous solution.

Independently, a spinning dope for the PLA layer having an average fiber diameter of 5 μm was prepared as a 9% PLA solution by blending 9% of the poly(lactic acid) having a molecular weight of 15×10⁴ to 17×10⁴ Da and 91% of chloroform. A spinning dope for the PLA layer having an average fiber diameter of 1 to 2 μm was prepared by blending 9% of the poly(lactic acid) having a molecular weight of 15×10⁴ to 17×10⁴ Da, 76% of chloroform, and 15% of methanol.

Using these spinning dopes, a guided bone regeneration membrane having a bilayer structure including an Si-PLA layer and a PLA layer having an average fiber diameter of 5 μm and another guided bone regeneration membrane having a bilayer structure including an Si-PLA layer and a PLA layer having an average fiber diameter of 1 to 2 μm were respectively manufactured through electrospinning. These membranes were manufactured under the same spinning conditions as below, except for the spinning dopes.

Spinning dope feed rate: about 0.05 ml/min, applied voltage: 20 kV, distance between the nozzle and collector: 15 cm, nozzle: laterally moves within a width of 15 cm at a rate of 15 cm/min, collector: conveyor-type collector (conveyor speed: 2 m/min), spinning time: about 60 minutes

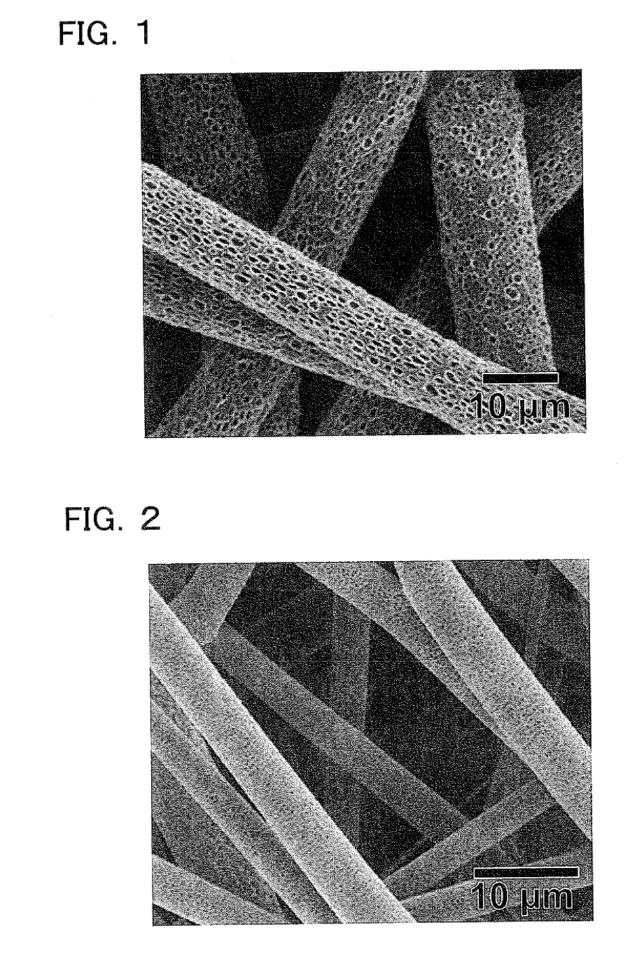

The microstructure of the Si-PLA layer as a first nonwoven fabric layer is shown in the scanning electron micrograph (SEM) of FIG. 1, demonstrating that the fibrous substance constituting the first nonwoven fabric layer has a diameter in the neighborhood of 10 μm.

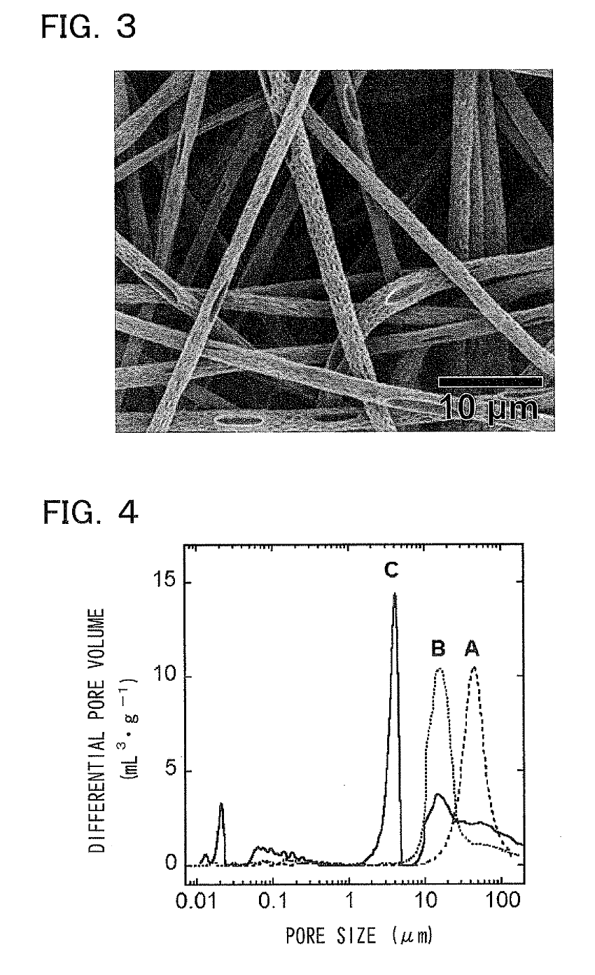

The microstructure of the PLA layer (average fiber diameter: 5 μm) as a second nonwoven fabric layer is shown in the scanning electron micrograph of FIG. 2, demonstrating that the fibrous substance constituting the second nonwoven fabric layer has a diameter in the neighborhood of 5 μm. The microstructure of the other PLA layer (average fiber diameter: 1 to 2 μm) as a second nonwoven fabric layer is shown in the scanning electron micrograph of FIG. 3, demonstrating that the fibrous substance constituting the second nonwoven fabric layer has a diameter in the neighborhood of 1 to 2 μm.

As demonstrated by FIGS. 1 to 3, a multiplicity of fibrous substance (fibers) contained in each nonwoven fabric layer show small variations in diameter and have substantially equivalent diameters. The porosities and pore sizes of Samples A, B, and C were measured with a mercury porosimeter (PoreSizer 9320, Shimadzu-Micrometrics) and are shown in FIG. 4. Samples A, B, and C correspond to the Si-PLA layer, the PLA layer (5 μm in diameter), and the PLA layer (1 to 2 μm in diameter) prepared in Example 1. Samples A, B, and C had porosities of 86%, 81%, and 70%, respectively, indicating no significant difference among the three samples. However, Samples A, B, and C differed in pore size and had pore sizes of 42 μm, 17 μm, and 11 μm, respectively.

FIG. 5 shows how deep cells invade into three nonwoven fabric layers composed of fibrous substances having different diameters. The symbol “*” means that there is a statistical difference in Student t-test (t<0.05). Specimens A, B, and C in FIG. 5 are PLA layers composed of fibrous substances having average diameters of 10 μm, 5 μm, and 1 to 2 μm, respectively, as in Example 1. FIG. 5 demonstrates that cell invasion of about 90 μm deep was observed on day 13 in Specimen A, indicating that Specimen A has a large fiber diameter and thereby has a large pore size, and this allows cells to invade into the nonwoven fabric layer. In contrast, no cell invasion was observed in Specimens B and C, indicating that Specimens B and C had small fiber diameters, thereby had pore sizes smaller than the sizes of cells per se, and this impeded the invasion of cells into the nonwoven fabric layers.

[Experimental Conditions for Cell Cultivation]

Cell type: Murine osteoblastic cells (MC3T3-E1 cells: Riken Institute of Physical and Chemical Research, Japan)

Cultivation using 24-well plate; Cell inoculation number: 1×10⁴ cells/well (Specimens A, 13, and C were placed respectively in a 24-well plate, exposed to α-MEM medium containing 10% fetal bovine serum for 1 hour, and then inoculated)

Culture medium: α-MEM medium (containing 10% fetal bovine serum)

Medium exchange: on the day following the inoculation, thereafter every other day

Cultivation: Each 1 ml of each suspension was added dropwise onto the specimen, followed by cultivation without any other treatment in an incubator at 37° C. in a 5% CO₂ atmosphere for 1, 6, and 13 days.

### Example 2

A Si—CaCO₃/PLA layer and a PLA layer were prepared as a first nonwoven fabric layer and a second nonwoven fabric layer, respectively. Specifically, a spinning dope for the first nonwoven fabric layer (Si—CaCO₃/PLA layer) was prepared as a spinning dope having a Si—CaCO₃ content of 13.0% and a PLA content of 8.7% by blending 1.5 g of Si—CaCO₃, 1.0 g of the poly(lactic acid) having a molecular weight of 15×10⁴ to 17×10⁴ Da, and 9.0 g of chloroform; and a spinning dope for the second nonwoven fabric layer (PLA layer) was prepared as a spinning dope having a PLA content of 9% by dissolving 1 g of the poly(lactic acid) having a molecular weight of 15×10⁴ to 17×10⁴ Da in 10.11 g of chloroform. Using these spinning dopes, a guided bone regeneration membrane having a bilayer structure of nonwoven fabrics was prepared through electrospinning.

[Si—CaCO₃/PLA Layer Preparation Conditions]

Spinning dope feed rate: about 0.24 ml/min, applied voltage: 20 kV, distance between the nozzle and collector: 15 cm, nozzle: laterally moves within a width of 15 cm at a rate of 10 cm/min, collector: conveyor-type collector (conveyor speed: 2 m/min), spinning time: about 180 minutes

[PLA Layer Preparation Conditions]

Spinning dope feed rate: about 0.05 ml/min, applied voltage: 20 kV, distance between the nozzle and collector: 15 cm, nozzle: laterally moves within a width of 15 cm at a rate of 15 cm/min, collector: conveyor-type collector (conveyor speed: 2 m/min), spinning time: about 180 minutes

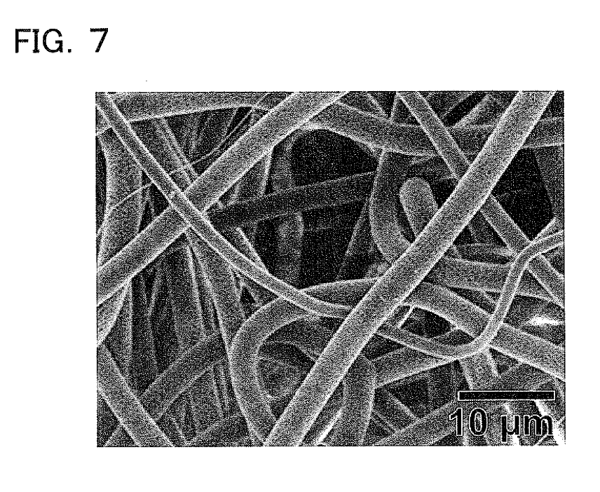

The microstructure of the Si—CaCO₃/PLA layer as the first nonwoven fabric layer is shown in the scanning electron micrograph (SEM) of FIG. 6, demonstrating that the fibrous substance constituting the first nonwoven fabric layer has a diameter in the neighborhood of 10 to 20 μm. The microstructure of the PLA layer as the second nonwoven fabric layer is shown in the scanning electron micrograph of FIG. 7, demonstrating that the fibrous substance constituting the second nonwoven fabric layer has a diameter in the neighborhood of 5 μm.

While the above description is of the preferred embodiments of the present invention, it should be appreciated that the invention may be modified, altered, or varied without deviating from the scope and fair meaning of the following claims.

What is claimed is:

1. A guided bone regeneration membrane having a bilayer structure comprising a first nonwoven fabric layer and a second nonwoven fabric layer, the first nonwoven fabric layer including a first fibrous substance containing a biodegradable resin as a principal component and further containing a siloxane, and the second nonwoven fabric layer including a second fibrous substance containing a biodegradable resin as a principal component,
wherein the first and second nonwoven fabric layers are both layers of electrospun nonwoven fabrics, and
wherein the second fibrous substance constituting the second nonwoven fabric layer has an average diameter smaller than that of the first fibrous substance constituting the first nonwoven fabric layer.

2. The guided bone regeneration membrane according to claim 1, wherein the second fibrous substance constituting the second nonwoven fabric layer has an average diameter of more than 0 μm and equal to or less than 5 μm.

3. The guided bone regeneration membrane according to claim 1, wherein the first fibrous substance constituting the first nonwoven fabric layer has an average diameter of 10 μm or more and 20 μm or less.

4. The guided bone regeneration membrane according to claim 1, wherein the first fibrous substance constituting the first nonwoven fabric layer further contains calcium carbonate fine particles and integrally contains the siloxane as dispersed in the calcium carbonate fine particles.

5. The guided bone regeneration membrane according to claim 1, wherein the biodegradable resin is a polylactic acid) or a copolymer thereof.

6. A method for manufacturing a guided bone regeneration membrane having a bilayer structure including a first nonwoven fabric layer and a second nonwoven fabric layer, the first nonwoven fabric layer including a first fibrous substance containing a biodegradable resin as a principal component and further containing a siloxane, and the second nonwoven fabric layer including a second fibrous substance containing a biodegradable resin as a principal component,
the method comprising the steps of:
forming the first nonwoven fabric layer through electrospinning; and
forming the second nonwoven fabric layer through electrospinning,
wherein the step of forming the second nonwoven fabric layer through electrospinning is performed so that the second fibrous substance constituting the second nonwoven fabric layer has an average diameter smaller than that of the first fibrous substance constituting the first nonwoven fabric layer.

7. The method according to claim 6, wherein the second fibrous substance constituting the second nonwoven fabric layer is formed so as to have an average diameter of more than 0 μm and equal to or less than 5 μm.

8. The method according to claim 6, wherein the first fibrous substance constituting the first nonwoven fabric layer is formed so as to have an average diameter of 10 μm or more and 20 μm or less.

9. The method according to claim 6, wherein a fibrous substance further containing calcium carbonate fine particles and integrally containing the siloxane as dispersed in the calcium carbonate fine particles is used as the first fibrous substance to constitute the first nonwoven fabric layer.

10. The method according to claim 6, wherein a poly(lactic acid) or a copolymer thereof is used as the biodegradable resin.
